(19) United States
(12) Patent Application Publication
(10) Pub. No.: US 2019/0276444 A1
(43) Pub. Date: Sep. 12, 2019

(54) BENZISOXAZOLES

(71) Applicant: Vanda Pharmaceuticals Inc., Washington, DC (US)

(72) Inventors: Joachim Nozulak, Heitersheim (DE); Hans Kalkman, Basel (CH)

(21) Appl. No.: 16/423,252

(22) Filed: May 28, 2019

Related U.S. Application Data
(63) Continuation of application No. 16/107,073, filed on Aug. 21, 2018, now abandoned.
Application No. 16/107,073 is a continuation of application No. 15/822,062, filed on Nov. 24, 2017, now abandoned.
Application No. 15/822,062 is a continuation of application No. 15/411,510, filed on Jan. 20, 2017, now abandoned.
Application No. 15/411,510 is a continuation of application No. 13/047,045, filed on Mar. 14, 2011, now abandoned.
Application No. 13/047,045 is a continuation of application No. 10/575,040, filed on Dec. 6, 2006, now abandoned.

(51) Int. Cl. C07D 413/04

(52) U.S. Cl. CPC C07D 413/04

(57) ABSTRACT

The present invention relates to novel fatty acid esters of the reversible Iloperidone metabolite P-88-8991, their preparation, their use as pharmaceuticals and pharmaceutical compositions containing them.

### CROSS-REFERENCE TO RELATED APPLICATIONS

This application is a continuation application of co-pending U.S. patent application Ser. No. 16/107,073, filed 21 Aug. 2018, which is a continuation of U.S. patent application Ser. No. 15/822,062, filed 24 Nov. 2017, which is a continuation of U.S. patent application Ser. No. 15/411,510, filed Jan. 20, 2017, which is a continuation of U.S. patent application Ser. No. 13/047,045, filed 14 Mar. 2011, which is a continuation of U.S. patent application Ser. No. 10/575,040, filed 6 Dec. 2006, which claims the benefit of PCT Application No. PCT/EP2004/010938, filed 30 Sep. 2004, which in turn claims the benefit of Great Britain Patent Application No. 0322994.5, filed 1 Oct. 2003, each of which is hereby incorporated herein.

### BACKGROUND OF THE INVENTION

The present invention relates to novel fatty acid esters of the reversible Iloperidone metabolite P-88-8991, their preparation, their use as pharmaceuticals and pharmaceutical compositions containing them.

Iloperidone is an atypical antipsychotic developed for the treatment of schizophrenia, having relevant affinity for noradrenergic, dopaminergic and serotoninergic receptors. See for example Richelson E. and Souder T., Life Sciences, 68:29-39 (2000).

### DETAILED DESCRIPTION

Iloperidone is metabolized in a reversible manner to P-88-8991 having the formula II

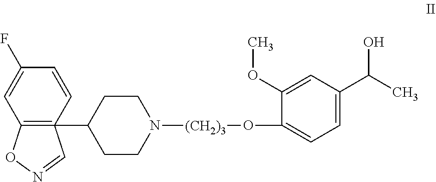

See for example Mutlib A E et al., Drug Metab. Dispos; 23(9):951-964 (1995). P-88-8991 has been shown to have plasma levels in human about 1.5 fold higher than the parent drug. It is roughly as active as Iloperidone.

More particularly, the invention relates to compounds of formula I

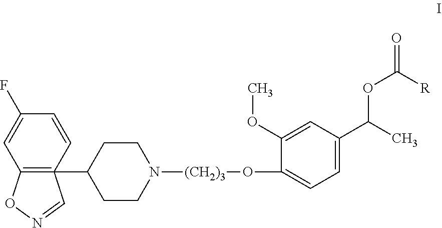

wherein R is (C₁₋₄₀)alkyl or (C₁₀)alkenyl, in free base or acid addition salt form.

On account of the asymmetrical carbon atom which is present in the compounds of formula I, the compounds may exist in optically active form or in form of mixtures of optical isomers, e.g. in form of racemic mixtures. All optical isomers and their mixtures including the racemic mixtures are part of the present invention.

In a further aspect, the invention provides a process for the production of the compounds of formula I and their salts, comprising the step of reacting the metabolite P-88-8991 of formula II with a compound of formula III

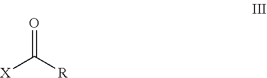

wherein R is as defined above and X is halogen, and recovering the so obtained compound of formula I in free base or acid addition salt form.

The reaction can be effected according to conventional methods, e.g. as described in Example 1.

In formula III, X is preferably chlorine or bromine.

Working up the reaction mixtures and purification of the compounds thus obtained may be carried out in accordance to known procedures.

Acid addition salts may be produced from the free bases in known manner, and vice-versa. Suitable acid addition salts for use in accordance with the present invention include for example the hydrochloride.

The starting compounds of formula II may be obtained by reducing Iloperidone of formula IV

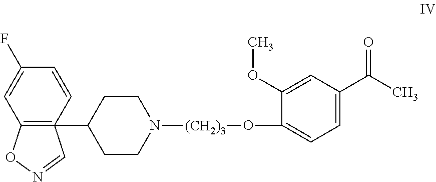

with an enantiomer of the boran complex of formula V

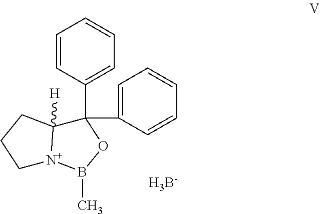

The compound of formula II (S)-1-(4-{3-[4-(6-fluoro-benzo[d]isoxazol-3-yl)-piperidin-1-yl]propoxy}-3-methoxy-enyl)-ethanol is obtained using the reagent (R)-2-methyl-CBS-oxazaborolidine-borane complex of formula Va

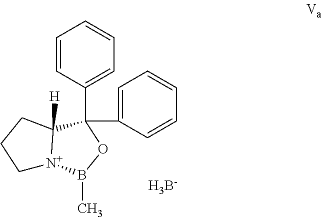

whereas the compound of formula II (R)-1-(4-{3-[4-(6-fluoro-benzo[d]isoxazol-3-yl)-piperidin-1-yl]propoxy}-3-methoxy-phenyl)-ethanol is obtained using the reagent (S)-2-methyl-CBS-oxazaborolidine-borane complex of formula Vb

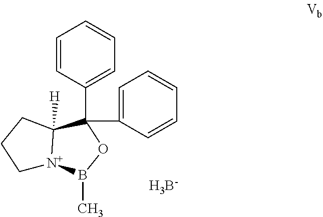

The reactions can be effected according to conventional methods, e.g. as described in Example 1.

The boran complexes used as starting materials can be produced from the corresponding compounds of formula VIa and VIb

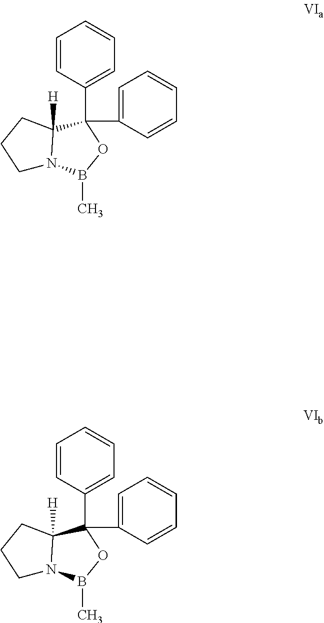

according to known procedures, e.g. as described in Example 1.

The starting materials of formulae VIa and VIb are known (for a review of these catalysts, see Corey, E. et al., Angew. Chem., Int. Ed. Engl. 1998, 37, 1986).

The compounds of formula I and their pharmaceutically acceptable acid addition salts, hereinafter referred to as agents of the invention, exhibit valuable pharmacological properties when tested in animals, and are therefore useful as pharmaceuticals.

In particular, the agents of the invention exhibit antipsychotic and anti-manic activity, as assessed in standard tests such as the amphetamine-induced hypermotility and the phencyclidine-induced hyperlocomotion tests.

The amphetamine-induced hypermotility test is performed according to the method described by Arnt J in Eur. J. Pharmacol. 283, 55-62 (1995). In this test, the agents of the invention significantly inhibit the amphetamine-induced locomotion of the animals at doses of about 0.01 to about 10 mg/kg s.c.

The phencyclidine-induced hyperlocomotion test is performed according to a rat adaptation of the method described by Gleason S D and Shannon H E in Psychopharmacol. 129, 79-84 (1997). In this test, the agents of the invention significantly block the phencyclidine-induced hyperlocomotion of the rats at doses of about 0.01 to about 10 mg/kg s.c.

The agents of the invention are therefore useful for the treatment of psychotic disorders such as schizophrenia and bipolar disorders.

It has been found that the agents of the invention are enzymatically metabolized into the active compound of formula II which is believed to be predominantly responsible of the in vivo activity in the above-mentioned tests. This ester cleavage has been found to proceed at slow rate. The agents of the invention are therefore of particular interest for use in pharmaceutical compositions aimed at providing a slow release of the compound of formula II.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 500, preferably from about 0.5 to about 100 mg/kg animal body weight. In larger mammals, for example humans, an indicated daily dosage is in the range from about 10 to about 2000, preferably from about 100 to about 1000 mg of an agent of the invention, conveniently administered, for example, in divided doses up to four times a day or in sustained release form.

The agent of the invention may be administered by any conventional route, preferably parenterally, for example in the form of injectable solutions or suspensions for intramuscular administration, or transdermally.

In accordance with the foregoing, the present invention also provides an agent of the invention, for use as a pharmaceutical, e.g. for the treatment of psychotic disorders.

The present invention furthermore provides a pharmaceutical composition comprising an agent of the invention in association with at least one pharmaceutical carrier or diluent. Such compositions may be manufactured in conventional manner. Unit dosage forms contain, for example, from about 0.25 to about 150, preferably from 0.25 to about 25 mg of a compound according to the invention.

Moreover the present invention provides the use of an agent of the invention, for the manufacture of a medicament for the treatment of psychotic disorders.

In still a further aspect the present invention provides a method for the treatment of psychotic disorders, in a subject in need of such treatment, which comprises administering to such subject a therapeutically effective amount of an agent of the invention.

The following examples illustrate the invention.

### Example 1

### (S)-(−)-Decanoic acid 1-(4-{3-[4-(6-fluoro-benzo[d]isoxazole-3-yl)-piperidine-1-yl]propoxy}-3-methoxy-phenyl)-ethyl ester

a) 200 ml of a solution of (3aR)-1-methyl-3,3-diphenyl-tetrahydro-pyrrolo[1 2-c][1,3,2)oxazaborole (1 M in toluene) is stirred at room temperature under nitrogen. 1.2 equivalent borane-dimethylsulfide complex is added with a syringe. The solution is stirred for 2 further hours at room temperature. The borane complex is then crystallised by addition of 4 vol dry hexane and cooling to −12° C. for 1.5 hour. The product is isolated by filtration in a sintered glass funnel and dried in vacuum at 40° C. The boran complex of (3aR)-1-methyl-3,3-diphenyl-tetrahydro-pyrrolo[1,2-c][1,3,2]oxazaborole is obtained (white crystals).

b) 56.36 g of boran complex of (3aR)-1-methyl-3,3-diphenyl-tetrahydro-pyrrolo[1,2-c] [1,3,2]oxazaborole (1 equivalent) is dissolved under nitrogen in methylenchioride, and the solution is cooled to 0° C. A 1M solution of 1-(4-{3-[4-(6-fluoro-benzo[d]isoxazol-3-yl)-piperidin-1-yl]-propoxy}-3-methoxy-phenyl)-ethanone (iloperidone; 1 equivalent) in methylenchloride is added via a dropping funnel over 90 minutes while the internal temperature is maintained at 0° C.±2° C. After the addition is complete, the mixture is stirred at 0° C. for 20 hours. The reaction mixture is then poured into precooled methanol (0-5° C.) during 1 hour. The solution is warmed to room temperature and stirred until the H₂ evolution ceases. The solution is concentrated by distillation and the residue dried in vacuum, treated with methanol and stirred for about 1 hour at 50° C. and an additional hour at 0° C. The product is isolated by filtration and dried under reduced pressure for 3 hours at 50° C. (S)-(−)-1-(4-{3-[4-(6-Fluoro-benzo[d]isoxazol-3-yl)-piperidin-1-yl]-propoxy}-3-methoxy-phenyl)-ethanol is obtained (white crystals).

[α]D²⁰−19.3° (c=1 in chloroform)

Mp: 138.2-138.8° C.

c) (S)-(−)-1-(4-{3-[4-(6-Fluoro-benzo[d]isoxazole-3-yl)-piperidine-1-yl]-propoxy)-3-methoxy-phenyl)-ethanol (8.57 g, 0.02 mol) is suspended in dichloromethane (150 mL) and pyridine (9.7 mL, 0.02 mol) is added. The reaction mixture is cooled and kept at 0° C.

Subsequently capric acid chloride (16.4 mL, 0.08 mol) is added slowly. The reaction mixture is further stirred at room temperature for 4 hours. The solution is then poured onto ice water and the liquid fractions are separated. The aqueous fraction is reextracted with methylenchloride. The combined organic fractions are dried and the solvent evaporated. The crude residue is purified by chromatography (neutral aluminum oxide, activity 3) to give (S)-(−)-decanoic acid 1-(4-{3-[4-(6-fluoro-benzo[d]isoxazole-3-yl)-piperidine-1-yl]-propoxy}-3-methoxy-phenyl)-ethyl ester as yellow oil with the optical rotation [α]—42.2° (c=0.5, methanol, T=20° C., 589 nm); e.e. 97.2%. The oxalate has a melting point of 99-100.3° C.

The following compounds of formula I are produced analogously to Example 1:

| | | | IR | | | |
|---|---|---|---|---|---|---|
| | | 13C-NMR | (C═0) | opt. | | |
| Example | R | C═O (ppm) | cm⁻¹ | Rot. | conc. | solv. |
| 2 | —(CH₂)₈—CH₃ | 173.150 | 1733 | −42.2 | 0.5 | Methanol |
| 3 | —CH₃ | 169.97 | 1728 | −43.9 | 0.5 | Methanol |
| 4 | —C₃H₇ | 172.47 | 1730 | −40.3 | 0.6 | Methanol |
| 5 | —(CH₂)₆—CH₃ | 173.105 | 1732 | −30.4 | 0.6 | Methanol |
| 6 | —(CH₂)₁₀—CH₃ | 173.161 | 1733 | −40.0 | 0.7 | Methanol |
| 7 | —(CH₂)₁₂—CH₃ | 173.138 | 1729 | −37.0 | 0.5 | Methanol |
| 8 | —(CH₂)₁₄—CH₃ | 173.179 | 1729 | −34.5 | 0.6 | Methanol |
| 9 | —CH₂—(CH₂—CH═CH)₆—CH₂—CH₃ | 172.392 | 1734 | | | |
| 10 | —(CH₂)₂—(CH₂—CH═CH)₅—CH₂—CH₃ | 172.008 | 1733 | | | |

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

1. A compound of Formula I 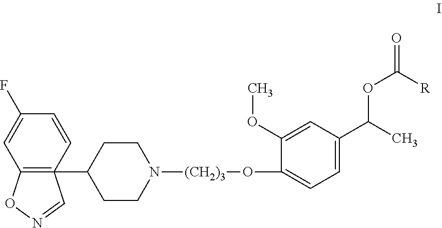
wherein R is (C₁₋₄₀)alkyl or (C₁₋₄₀)alkenyl, in free base or acid addition salt form, and wherein the acid addition salt form is a pharmaceutically acceptable acid addition salt form.

2. A method for the production of the compounds of formula I 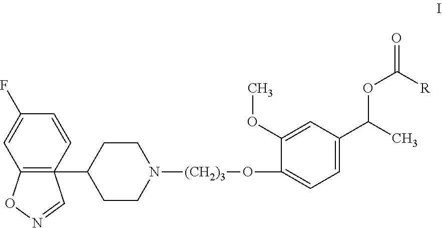
wherein R is (C₁₋₄₀)alkyl or (C₁₋₄₀)alkenyl, and their salts, the method comprising:
reacting a compound of formula II 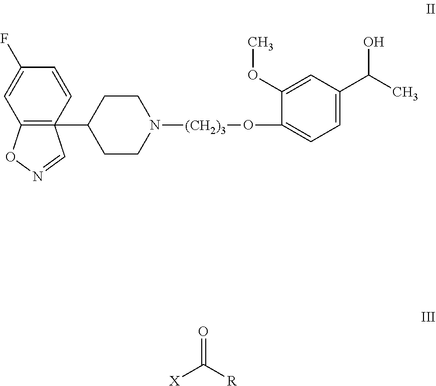
with a compound of formula III
wherein R is (C₁₋₄₀)alkyl or (C₁₋₄₀)alkenyl and X is halogen; and
recovering the resulting compound in free base or acid addition salt form.

3. The compound of claim 1, which is decanoic acid 1-(4-{3-[4-(6-fluoro-benzo[d]isoxazole-3-yl)-piperidine-1-yl]propoxy}-3-methoxy-phenyl)-ethyl ester.

4. The compound of claim 1, which is (S)-(−)-decanoic acid 1-(4-{3-[4-(6-fluoro-benzo[d]isoxazole-3-yl)-piperidine-1-yl]propoxy}-3-methoxy-phenyl)-ethyl ester.

5. The compound of claim 1, wherein R is selected from —(CH₂)₈—CH₃, —(CH₂)₆—CH₃, —(CH₂)₁₀—CH₃, —(CH₂)₁₂—CH₃, —(CH₂)₁₄—CH₃, —CH₂—(CH₂—CH═CH)₆—CH₂—CH₃, or —(CH₂)₂—(CH₂—CH═CH)₅—CH₂—CH₃.

6. A pharmaceutical composition for the treatment of a psychotic disorder comprising a compound of Formula I,
wherein R is (C₁₋₄₀)alkyl or (C₁₋₄₀)alkenyl, in free base or acid addition salt form; and
a pharmaceutical carrier or diluent.

7. The pharmaceutical composition of claim 6, wherein the compound of Formula I is decanoic acid 1-(4-{3-[4-(6-fluoro-benzo[d]isoxazole-3-yl)-piperidine-1-yl]propoxy}-3-methoxy-phenyl)-ethyl ester.

8. The pharmaceutical composition of claim 6, wherein the compound of Formula I is (S)-(−)-decanoic acid 1-(4-{3-[4-(6-fluoro-benzo[d]isoxazole-3-yl)-piperidine-1-yl]propoxy}-3-methoxy-phenyl)-ethyl ester.

9. The pharmaceutical composition of claim 6, wherein R is selected from —(CH₂)₈—CH₃, —(CH₂)₆—CH₃, —(CH₂)₁₀—CH₃, —(CH₂)₁₂—CH₃, —(CH₂)₁₄—CH₃, —CH₂—(CH₂—CH═CH)₆—CH₂—CH₃, or —(CH₂)₂—(CH₂—CH═CH)₅—CH₂—CH₃.

10. The pharmaceutical composition of claim 6, wherein the compound of Formula I is present in a unit dosage form from about 0.25 to about 150 mg.

11. The pharmaceutical composition of claim 6, wherein the compound of Formula I is present in a unit dosage form from about 0.25 to about 25 mg.

12. The pharmaceutical composition of claim 6, wherein the psychotic disorder is schizophrenia or a bipolar disorder.
